(19) United States
(12) Patent Application Publication
(10) Pub. No.: US 2023/0014400 A1
(43) Pub. Date: Jan. 19, 2023

(54) DEVICE, SYSTEM AND METHOD FOR VERIFIED SELF-DIAGNOSIS

(71) Applicant: Dynamic Century Holdings Limited, Hong Kong (CN)

(72) Inventors: King Shiu Kelvin WU, Hong Kong (CN); Snir ZANO, Omer (IL); Inon AXELROD, Atlit (IL)

(73) Assignee: Dynamic Century Holdings Limited, Hong Kong (CN)

(21) Appl. No.: 17/863,636

(22) Filed: Jul. 13, 2022

Related U.S. Application Data
(60) Provisional application No. 63/221,958, filed on Jul. 15, 2021.

(51) Int. Cl. G16H 10/40; G16H 40/60; G06V 40/16

(52) U.S. Cl. CPC G16H 10/40; G16H 40/60; G06V 40/171

(57) ABSTRACT

Methods and systems are provided for verifying results of a self-test by a subject using a test kit. The subject's identity may be verified, for example using AI-assistant facial recognition and/or data obtained from scanned government issued documents of the subject. Images obtained while the test is conducted may be used to determine if the test is conducted properly and images obtained of the completed self-test may be analyzed to determine the test results. Test results, verified as belonging to the subject and having been correctly performed, may be uploaded to a remote database as part of a health “passport” program.

### CROSS-REFERENCE TO RELATED APPLICATIONS

The present application claims the benefit of U.S. Provisional Application No. 63/221,958, filed Jul. 15, 2021, which is incorporated herein by reference in its entirety.

### FIELD OF THE INVENTION

The present invention relates to devices and kits for self-testing, methods of using them and systems for providing verification of self-tests, such as home testing for Covid-19 or other diseases and health conditions.

### BACKGROUND OF THE INVENTION

Widespread adoption of home testing for Covid-19 and other diseases and health conditions has brought with it a host of related problems, including ensuring that the proper test is delivered to the intended test subject, that the subject self-administers the intended test properly, and that the results are securely delivered to the intended recipients.

### SUMMARY OF THE INVENTION

These objects and others are achieved with the systems and methods of the present invention which moreover provide that the results of such testing may be recorded uniformly and systematically throughout a population, for example providing a “health passport” indicating the health status of an individual in a universally recognized format (where permitted by law).

Some embodiments of the present invention may provide a method of verifying a self-test by a subject using a test kit, the method comprising: obtaining, by a computing device, data identifying of the subject; obtaining, by the computing device, data identifying the test kit; verifying, by the computing device, that the identified subject is the subject utilizing the test kit; obtaining, by the computing device, an in-process image of the self-test being performed and determining from the in-process image that the self-test has been properly performed; obtaining, by the computing device, a results image of the self-test after the self-test has been performed and from said results image obtain a test result; and registering in memory, by the computing device, the obtained test result.

In some embodiments, identifying the subject comprises: obtaining, by a camera associated with the computing device, a first image of an identification document comprising a visual representation of a face of the subject, obtaining, by the camera, a second image of the subject; and identifying, by the computing device, the subject based on a comparison of the first image and the second image.

In some embodiments, the method further comprising verifying, by the computing device, the identification document based on a comparison of data extracted from the first image of the identification document and a reference dataset.

In some embodiments, verifying that the identified subject is the subject utilizing the test kit comprises: obtaining, by the camera, a third image comprising a visual representation of the face of the subject and of a component of the test kit, and determining, by the computing device, that the identified subject is the subject utilizing the test kit based on the third image and at least one of the first image, the second image, the reference dataset, or any combination thereof.

In some embodiments, the method further comprising verifying, by the computing device, that the subject is properly utilizing the test kit.

In some embodiments, verifying that the subject is properly utilizing the test kit comprises: detecting, by the computing device, a specified location on the face of the subject in the third image, detecting, by the computing device, a position of the component of the test kit with respect to the specified location on the face of the subject in the third image, and determining, by the computing device, that the subject is properly utilizing the test kit based on the position of the component of the test kit with respect to the specified location on the face of the subject in the third image.

In some embodiments, obtaining data identifying the test kit comprises: obtaining, by a camera associated with the computing device, a third image of an optical label present on one or more components of the test kit, the optical label containing identification information of the test kit, and identifying, by the computing device, the test kit based on the obtained image and a reference dataset.

In some embodiments, obtaining the test result from the test kit and verifying that the test kit is the identified test kit comprising: obtaining, by the camera, a fifth image comprising a visual representation of a test result and of the optical label being presented on a component of the test kit, and determining, by the computing device, that the test kit is the identified test kit based on the obtained image and the reference dataset.

n some embodiments, registering the obtained test result with the authorized third-party comprising sending, by the computing device, to the authorized party the obtained image comprising the visual representation of the test result and of the optical label being presented on the component of the test kit.

Some embodiments of the present invention may provide a method of self-diagnosing of a subject using a test kit comprising a sample collecting tool and a testing tool, the method comprising: obtaining, by a camera associated with a computing device, a first image of an identification document comprising a visual representation of a face of the subject, obtaining, by the camera, a second image comprising a visual representation of the face of the subject, identifying, by the computing device, the subject based on the first image and the second image, obtaining, by the camera, a third image of an optical label being presented on one or more components of the test kit, the optical label containing identification information of the test kit, identifying, by the computing device, the test kit based on the third image and a reference dataset, obtaining, by the camera, a fourth image comprising a visual representation of the face of the subject and of the sample collecting tool of the test kit, determining, by the computing device, that the identified subject is the subject utilizing the sample collecting tool of the test kit based on the fourth image and at least one of the first image or the second image, detecting, by the computing device, a specified location on the face of the subject in the fourth image, detecting, by the computing device, a position of the sample collecting tool of the test kit with respect to the specified location on the face of the subject in the fourth image, determining, by the computing device, that the identified subject is properly utilizing the sample collecting tool of the test kit based on the position of the sample collecting tool with respect to the specified location on the face of the subject in the fourth image, obtaining, by the camera, a fifth image comprising a visual representation of a test result and of the optical label being presented on the testing tool of the test kit, determining, by the computing device, that the testing tool belongs to the identified test kit based on the fifth image and the reference dataset, and registering the test result with an authorized third party by the computing device.

In some embodiments, the test kit is an antigen test kit.

In some embodiments, the test kit is a serological test kit.

In some embodiments, the test kit is a polymerase chain reaction (PCR) test kit.

Some embodiments of the present invention may provide a system for verifying a self-test by a subject, comprising: a test kit adapted to provide a readable display of test results; and a computing device having a processor configured to obtain identifying information from a subject to verify the subject's identity; obtain identifying information from the test kit to verify the test kit and associate the test kit with the subject; obtain information about performance of the test kit; and read the readable display of test results of the test kit.

In some embodiments, the test kit includes a bar code readable by the computing device to verify the test kit.

In some embodiments, the computing device comprises a scanner adapted to scan government issued documentation of the subject to extract identifying information of the subject, and/or an image of the subject.

In some embodiments, the processor is configured to compare the identifying information obtained from the subject with a remote dataset.

In some embodiments, the computing device comprises a camera adapted to obtain an image of the subject's face, and wherein identifying information from a subject comprises facial features of the subject identified in the image of the subject's face.

In some embodiments, the computing device comprises a camera adapted to obtain an image of the subject's face during performance of the test and verify correct performance of the test from an image of the subject's face during performance of the test.

In some embodiments, the test kit comprises a lateral flow test strip forming a visible band on the strip when analyte is detected and the processor is configured to determine a positive or negative a test result from an image of the test strip.

In some embodiments, the teste kit comprises a nasal swab and the processor is configured to identify indicia on the nasal swab in relation to facial features of the subject when the self-test is being performed.

In some embodiments, the computing device is smart phone.

In some embodiments, the computing device and test kit are associated in a kiosk location.

### BRIEF DESCRIPTION OF THE DRAWINGS

In order for the present invention to be better understood and for its practical applications to be appreciated, the following Figures are provided and referenced hereinafter. It should be noted that the Figures are given as examples only and in no way limit the scope of the invention. Like components are denoted by like reference numerals.

FIG. 1 shows a block diagram of an exemplary computing device, according to some embodiments of the invention;

FIGS. 2A-2C depict modes of identifying a computing device within a system according to embodiments of the invention;

FIGS. 3A-3C depict modes of identifying an individual subject within a system according to embodiments of the invention;

FIGS. 4A-4C depict modes of identifying a test kit within a system according to embodiments of the invention;

FIGS. 5A-5F depict modes of monitoring a self-test and transmitting the results; and

FIG. 6 is a schematic diagram of general system architecture according to an embodiment of the invention.

### DETAILED DESCRIPTION OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other non-transitory storage medium (e.g., a memory) that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, the conjunction “or” as used herein is to be understood as inclusive (any or all of the stated options). A “kit” is a group of disparate objects assembled for a purpose. For example a “test kit” disclosed herein may contain a swab, a vial, chemical reagents and printed instructions or a label all associated with one another for the purpose of conducting a medical test.

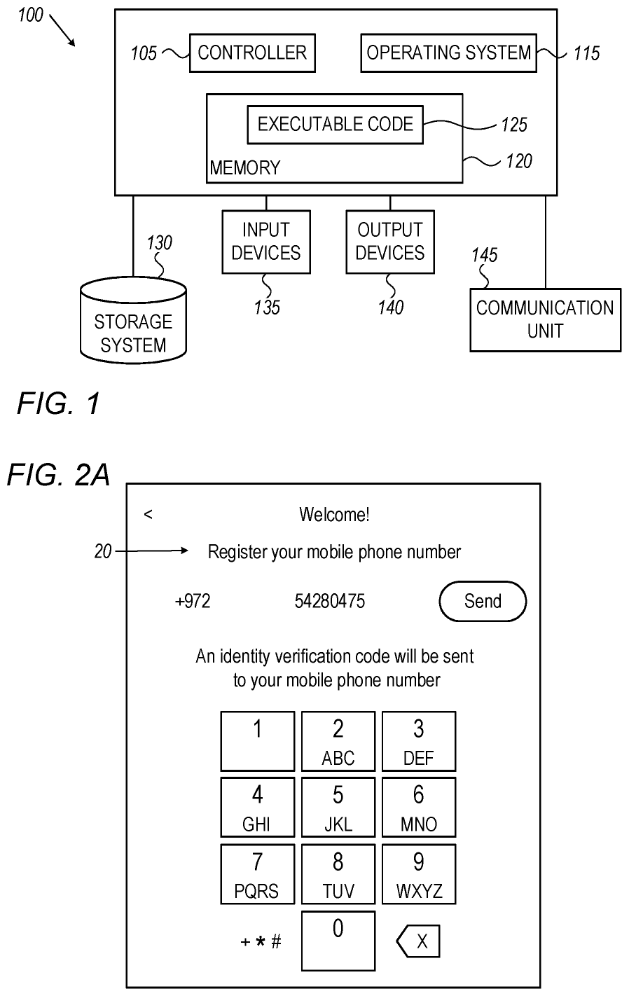

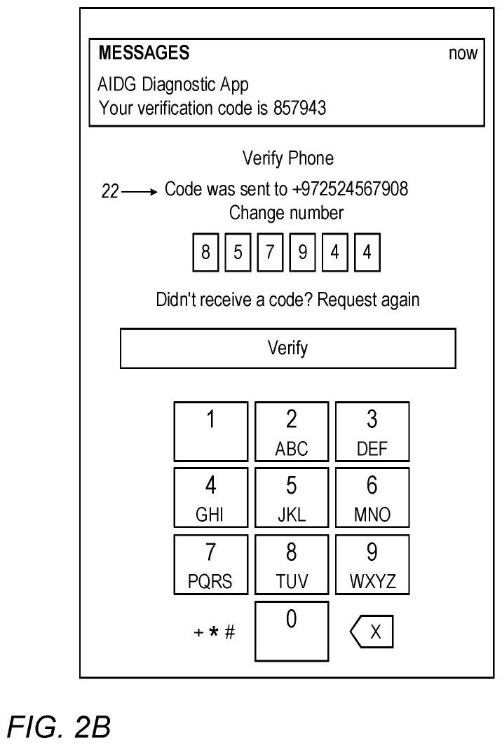

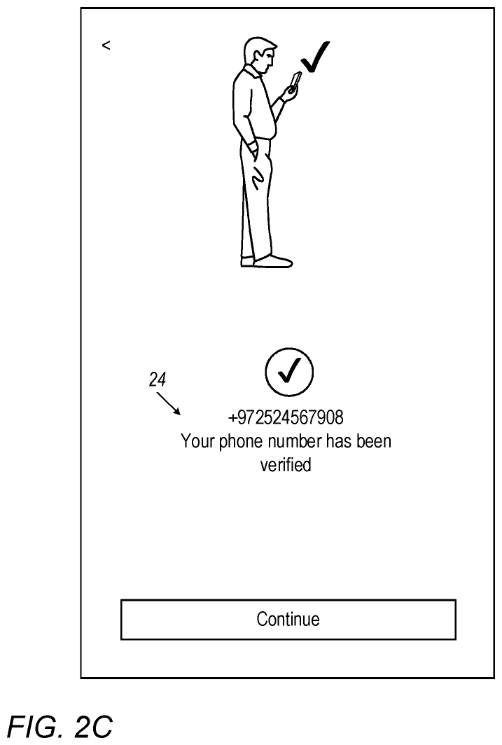

Reference is made to FIG. 1, which is a schematic block diagram of an example computing device, according to some embodiments of the invention. Computing device 100 may include a controller or processor 105 (e.g., a central processing unit processor (CPU), a chip or any suitable computing or computational device), an operating system 115, memory 120, executable code 125, storage 130, input devices 135 (e.g. a keyboard or touchscreen), and output devices 140 (e.g., a display), a communication unit 145 (e.g., a cellular transmitter or modem, a Wi-Fi communication unit, or the like) for communicating with remote devices via a communication network, such as, for example, the Internet. Controller 105 may be configured to execute program code to perform operations described herein. The system described herein may include one or more computing device(s) 100, for example, to act as the various devices or the components shown in FIG. 2. For example, system 200 may be, or may include computing device 100 or components thereof.

Operating system 115 may be or may include any code segment (e.g., one similar to executable code 125 described herein) designed and/or configured to perform tasks involving coordinating, scheduling, arbitrating, supervising, controlling or otherwise managing operation of computing device 100, for example, scheduling execution of software programs or enabling software programs or other modules or units to communicate.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of similar and/or different memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of operating system 115. For example, executable code 125 may be a software application that performs methods as further described herein. Although, for the sake of clarity, a single item of executable code 125 is shown in FIG. 1, a system according to embodiments of the invention may include a plurality of executable code segments similar to executable code 125 that may be stored into memory 120 and cause controller 105 to carry out methods described herein.

Storage 130 may be or may include, for example, a hard disk drive, a universal serial bus (USB) device or other suitable removable and/or fixed storage unit. In some embodiments, some of the components shown in FIG. 1 may be omitted. For example, memory 120 may be a non-volatile memory having the storage capacity of storage 130. Accordingly, although shown as a separate component, storage 130 may be embedded or included in memory 120.

Input devices 135 may be or may include a keyboard, a touch screen or pad, one or more sensors or any other or additional suitable input device. Any suitable number of input devices 135 may be operatively connected to computing device 100. Output devices 140 may include one or more displays or monitors and/or any other suitable output devices. Any suitable number of output devices 140 may be operatively connected to computing device 100. Any applicable input/output (I/O) devices may be connected to computing device 100 as shown by blocks 135 and 140. For example, a wired or wireless network interface card (NIC), a universal serial bus (USB) device or external hard drive may be included in input devices 135 and/or output devices 140.

Embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein. For example, an article may include a storage medium such as memory 120, computer-executable instructions such as executable code 125 and a controller such as controller 105. Such a non-transitory computer readable medium may be for example a memory, a disk drive, or a USB flash memory, encoding, including or storing instructions, e.g., computer-executable instructions, which when executed by a processor or controller, carry out methods disclosed herein. The storage medium may include, but is not limited to, any type of disk including, semiconductor devices such as read-only memories (ROMs) and/or random-access memories (RAMs), flash memories, electrically erasable programmable read-only memories (EEPROMs) or any type of media suitable for storing electronic instructions, including programmable storage devices. For example, in some embodiments, memory 120 is a non-transitory machine-readable medium.

A system according to embodiments of the invention may include components such as, but not limited to, a plurality of central processing units (CPUs), a plurality of graphics processing units (GPUs), or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components. In some embodiments, a system may include or may be, for example, a personal computer, a desktop computer, a laptop computer, a workstation, a server computer, a network device, or any other suitable computing device. For example, a system as described herein may include one or more facility computing device 100 and one or more remote server computers in active communication with one or more facility computing device 100 such as computing device 100, and in active communication with one or more portable or mobile devices such as smartphones, tablets and the like. Several programming modules are described below, which are portions of code or routines that may be assembled together in different configurations to make a computer program or system.

In some instances involving facial recognition and analysis of test results, one or more neural networks may be used. Neural networks (NN) or connectionist systems are computing systems inspired by biological computing systems, but operating using manufactured digital computing technology. NNs are made up of computing units typically called neurons (which are artificial neurons or nodes, as opposed to biological neurons) communicating with each other via connections, links or edges. In common NN implementations, the signal at the link between artificial neurons or nodes can be for example a real number, and the output of each neuron or node can be computed by function of the (typically weighted) sum of its inputs, such as a rectified linear unit (ReLU) function. NN links or edges typically have a weight that adjusts as learning proceeds. The weight increases or decreases the strength of the signal at a connection. Typically, NN neurons or nodes are divided or arranged into layers, where different layers can perform different kinds of transformations on their inputs and can have different patterns of connections with other layers.

NN systems can learn to perform tasks by considering example input data, generally without being programmed with any task-specific rules, being presented with the correct output for the data, and self-correcting, or learning.

Various types of NNs exist. For example, a convolutional neural network (CNN) can be a deep, feed-forward network, which includes one or more convolutional layers, fully connected layers, and/or pooling layers. CNNs are particularly useful for visual applications. Other NNs can include for example transformer NNs, useful for speech or natural language applications, and long short-term memory (LSTM) networks.

In practice, a NN, or NN learning, can be simulated by one or more computing nodes or cores, such as generic central processing units (CPUs, e.g. as embodied in personal computers) or graphics processing units (GPUs such as provided by Nvidia Corporation), which can be connected by a data network. A NN can be modelled as an abstract mathematical object and translated physically to CPU or GPU as for example a sequence of matrix operations where entries in the matrix represent neurons (e.g. artificial neurons connected by edges or links) and matrix functions represent functions of the NN.

Typical NNs can require that nodes of one layer depend on the output of a previous layer as their inputs. Current systems typically proceed in a synchronous manner, first typically executing all (or substantially all) of the outputs of a prior layer to feed the outputs as inputs to the next layer. Each layer can be executed on a set of cores synchronously (or substantially synchronously), which can require a large amount of compute power, on the order of 10s or even 100s of Teraflops, or a large set of cores. On modern GPUs this can be done using 4,000-5,000 cores.

Using a system for self-testing of a subject according to embodiments of the invention involves a computing device, such as a smart phone having an installed program information, such as an “app,” adapted to perform registration of the device to be used in connection with the system; acquire a video image of the subject; acquire a video image of documentation of the subject; perform image analysis of the video image of the subject and the documentation to confirm the identity of the subject.

In embodiments, the system is configured to identify a test apparatus or kit, such as by “reading” indicia associated with the apparatus; transmit instructions for performing the self-test to the subject, and in embodiments perform confirmation that the self-test is properly administered, for example by acquiring a video image of the self-test being performed and performing image analysis, or alternatively, by acquiring data input by the user sufficient to establish correct completion of the self-test. In embodiments, the processor is configured read indicia on the self-test to determine results and to transmit the results of the test in a format suitable to provide standardized documentation of the subject's test results. In embodiments, the processor is adapted to acquire and integrate third party input at different stages of the process, as described below.

In FIGS. 2A-2C a subject having an installed app on a smart phone registers the device in response to prompts 20, 22, 24 to identify the device with that unique number as belonging to the subject. The steps of identifying a device using a token or other mechanism are known in the art and are not elaborated herein.

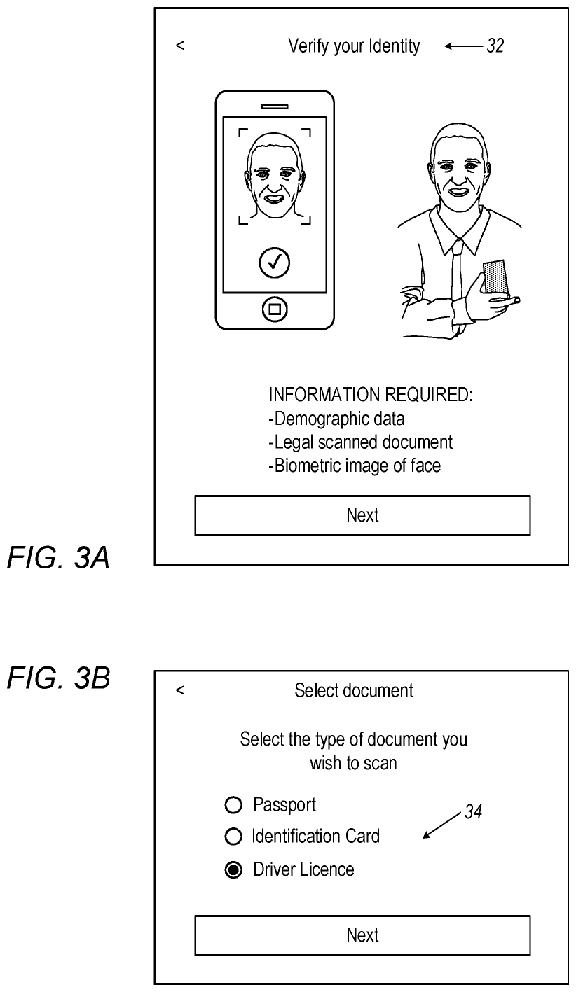

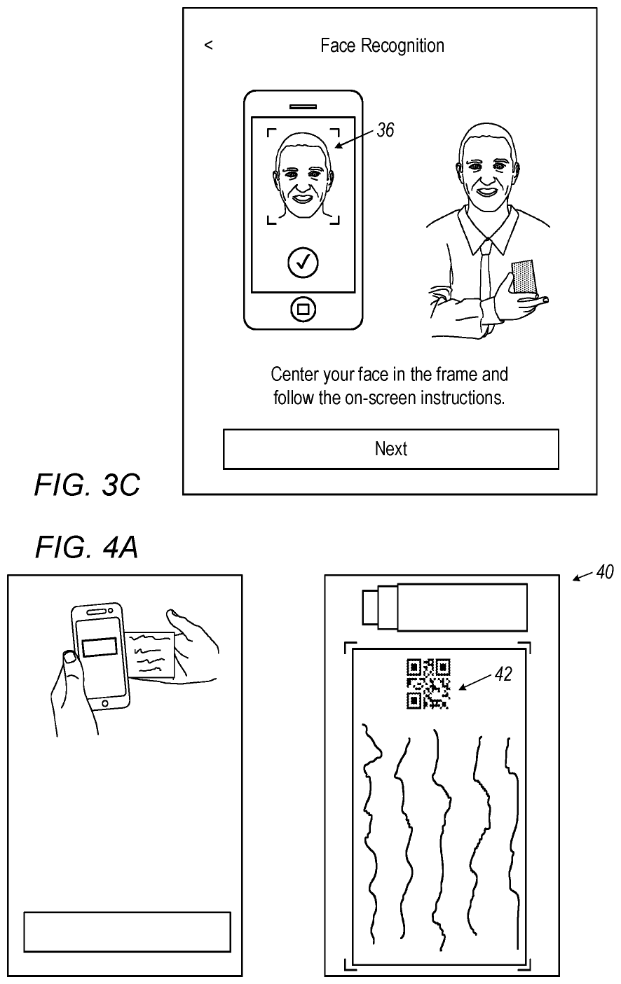

FIG. 3A is an example of a screen prompt 32 adapted to be displayed to the subject depicting minimum information and documentation required to successfully complete a self-test and obtain verification according to embodiments of the invention. Information required to verify a subject's identity may include demographic data input by the subject on the device, obtained previously, or extracted from the subject's identification documents; a biometric image of the subject's face acquired by the device; and a scanned image of government-issued identification (passport, license, etc.) As depicted in FIG. 3B, acceptable government-issued identification may be selected by the subject from a predetermined list 34, so that a processor may extract image data or other data from known locations on the identification. As known in the art, the device may prompt a user to scan the government-issued identification. As known in the art, the device may present a form allowing a user to input demographic data. The data, extracted by the processor or entered by the subject, may be compared with information on a remote database.

In embodiments, a document recognition module (DRM) may involve image recognition and machine learning technologies to identify the type of identification document provided by the user (e.g., identification card, driver's license, passport, etc). Used together with a mobile camera, the DRM supports the task of acquiring a quality image of an authenticated identification document. The document may need to be an accepted form of a recognized identification document that includes a profile image and other commonly known identification markings.

In combination with the DRM, an accredited profile module (APM) may identify the coordinate position (corners) of a profile image containing a front facing facial image that is integrated as part of the recognized identification document and excerpts the photo as an approved and accredited facial image of the registrant. Together with the DRM, a document optical character recognition (OCR) module may identify data printed on a recognized identification document and excerpt relevant data required for registration such as name, id number, birthdate, address, etc. Using (OCR), images of printed text may be converted into machine-encoded text data. Data may be presented for the registrant's confirmation by method of a registration form prior to storing the data. This method of filling a registration form by OCR may reduce the number of typos or mistakenly filled data. The registrant may still be required to approve the authenticity of the registered data by form of an active user action.

As depicted in FIG. 3C, the system may prompt a subject to provide an image. The application may utilize a mobile camera, such as on a user smartphone, to acquire a quality image of a camera view in order to collect visual information. With the help of on-screen feedback the module may guide the user to the correct position and zoom of the camera lens (e.g., angle, distance, focus and exposure) in reference to an object. Adjustments are made by moving the camera lens relatively to the object or moving the object relatively to the position of the camera lens.

The user can actively click to acquire an image or the snapshot can be triggered automatically when the module confirms that the camera is in the required position and that the resulting image recognition is to the requirement. Facial recognition software may be used to compare the image with an image of the subject's face obtained by scanning the government-issued identification. Information extracted from the government-issued identification may be compared with information entered on a form or the visual or text data of may be compared with information present in a reference dataset pertaining to the subject. The system may allow communication with a “back-office” at any stage so the subject can obtain live assistance in verifying themselves as an authorized user. For example, facial recognition software may fail to match the video image of the subject with the government-issued identification photograph. The subject may seek to have live confirmation of their identity, and one or more processors of the device may initiate contact with the live confirmation.

Face comparison is the procedure of comparing a particular face with one or more others and measuring the similarity value. Using the Amazon Rekognition Image CompareFaces API, the likelihood that two faces belong to the same person may be measured.

The API compares the faces found in the source and target images and returns a similarity value for each pair. The service also returns the frame and confidence level for each detected face for defining an authenticity threshold. Face comparison can be used to identify users from previously saved photos in near real time

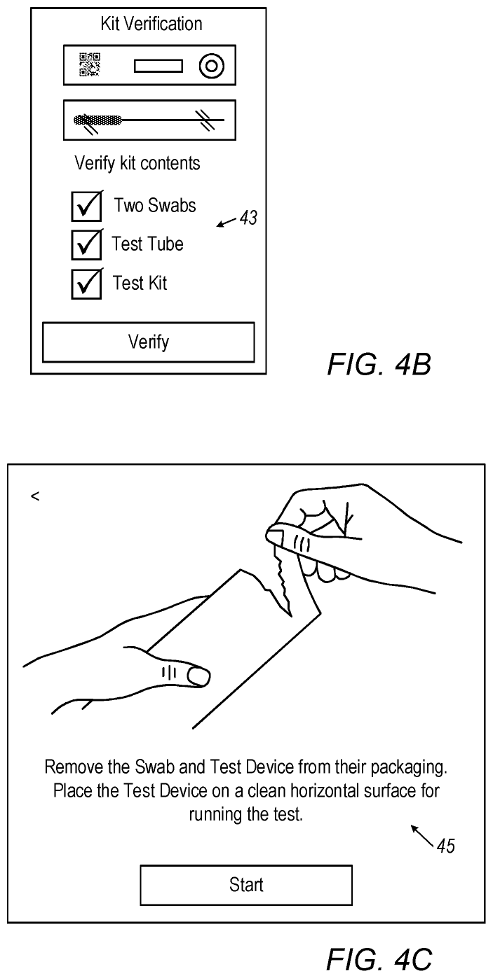

FIG. 4A depicts a test kit label 40 having a QR code 42 and a step of identifying a test kit by scanning the QR code according to embodiments of the invention. A subject using the self-test scans the QR code to verify, by a processor, what the test is and to initiate, by a processor, a sequence for the processor to display steps to the subject appropriate for the test. In embodiments, the test kit is associated with the subject, i.e., the self-test was ordered by or for the subject, and the processor may indicate that the kit is matched with the subject. In embodiments, a kit includes a device for obtaining a biological sample from a subject. For example, in embodiments a kit includes a nasal swab for obtaining a sample from a subject's nasal passageway, as known in the art. In embodiments, other biological samples may be obtained, including without limitation skin, tissue, saliva, nails, hair, or blood, utilizing a device for obtaining such samples, as known in the art. Embodiments of the invention are adapted for home use. In other embodiments test results are shipped to a designated laboratory. In embodiments, kiosk stations may be employed as designated test sites, with the use of the kiosk stations as post boxes where all samples in the kiosk are collected to be sent to a laboratory for determining results.

In embodiments, the computing device may verify that the subject is properly utilizing the test kit using different functionalities, alone or in combination, for example:

The subject may select kit type, manually or by scanning a QR code with the device, which prompts one or more instructional screens or videos appropriate for that test to play on the device. According to embodiments, a user can consult is a physician by means of a videoconference. In embodiments, such interaction with a physician may be provided for in a method of using the test kit. For example, the user may be prompted by the app contact a physician with questions. Some types of results by law can only be released during a conference meeting with the doctor so that they can explain the results to the patient to prevent a patient interpreting the result incorrectly. Such result may be released in the app only after a videoconference meeting with a doctor. In embodiments, a user receives results within the app and the user is able to further consult with a doctor by videoconference for further testing and treatment.

The test may be timed by an internal clock functionality such that failure to complete the test within an allowed time frame prevents results from being automatically verified.

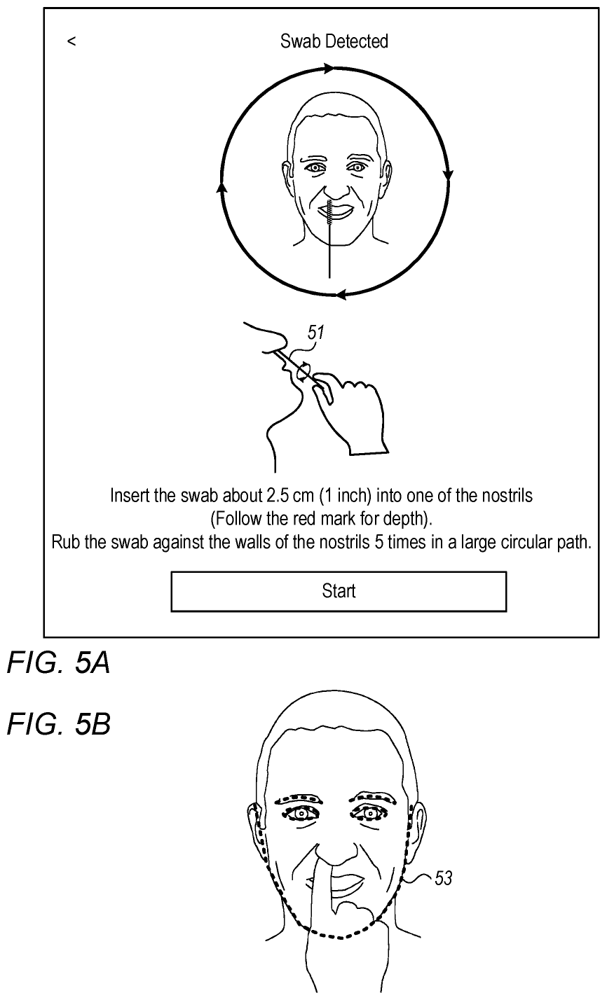

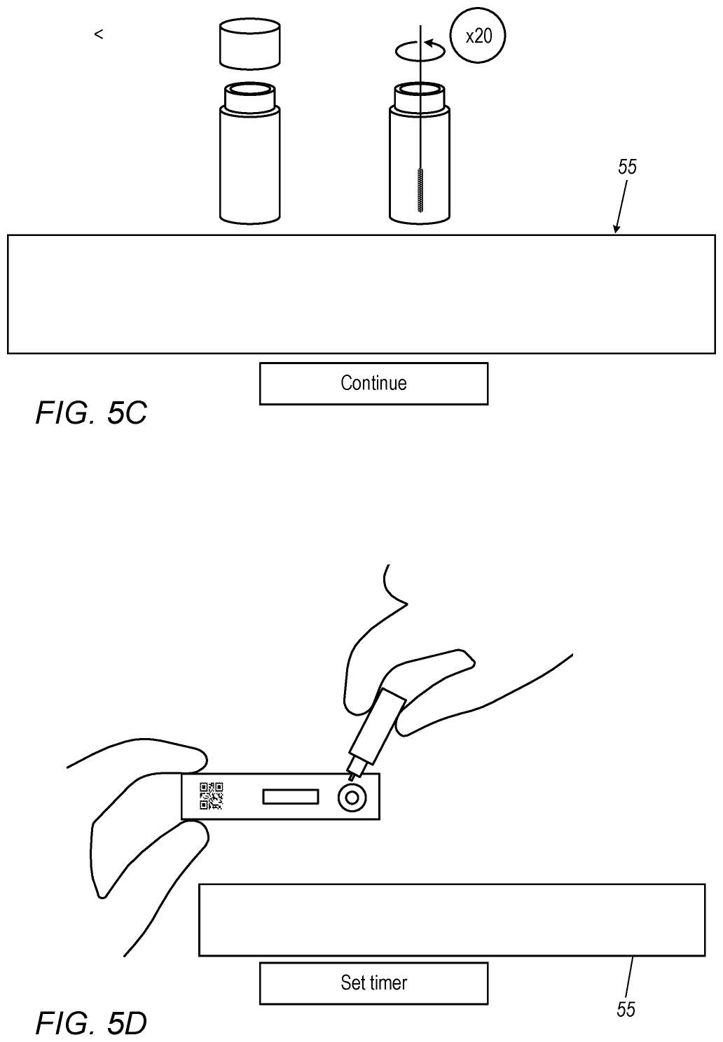

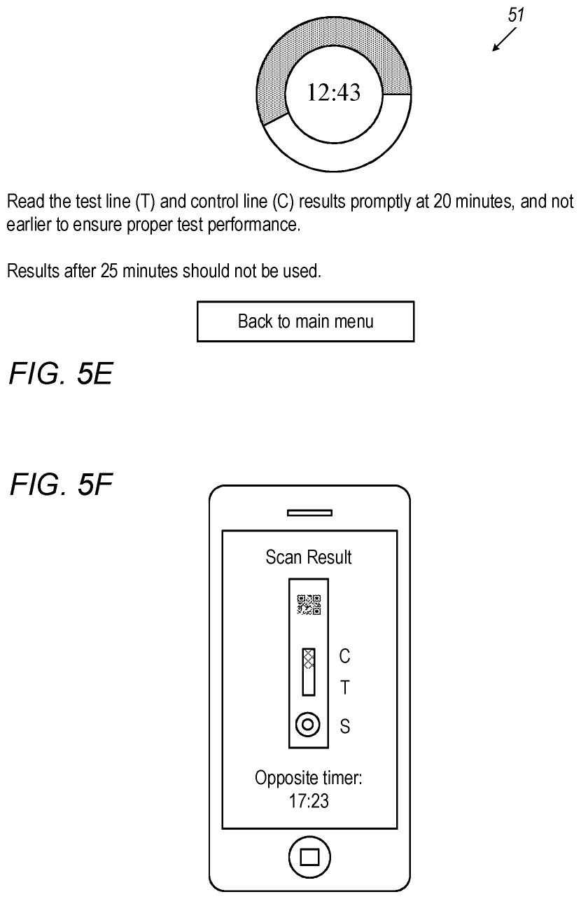

Video of the self-test may be acquired by the device to verify execution of the test. Referring to FIG. 5A the device may use artificial intelligence to determine whether a swab is being correctly inserted into the subject's nose. In FIG. 5B, the device processor may identify landmarks on a subject's face to determine if a subject is correctly performing a test or deliberately causing a test to fail.

Verifying that the subject is properly utilizing the test kit may involve detecting, by the computing device, a specified location on the face of the subject in an image obtained during execution of the test; determining a position of the component of the test kit with respect to the specified location on the face of the subject in the image, and determining, by the computing device, that the subject is properly utilizing the test kit based on the position of the component of the test kit with respect to the specified location on the face of the subject in the third image. For example, a processor using artificial intelligence algorithms may determine that a swab is placed far enough into the subject's nasal passage. Users are required to take a snapshot of the test kit and submit the test for further analysis. Analysis is done by image recognition and machine learning algorithms in order to obtain a test result.

The algorithm for evaluating an image of test results may incorporate PyTorch, which is an open-source machine learning framework which is based on Torch. PyTorch is used for model development, especially, detecting tests and to classify them. Default packages such as Numpy, Maths and OpenCV may be used for denoising and preprocessing images. Input format claims image of the test kit then the module gives output of the detected result. In order to implement the ability to analyze test results, hundreds of collected photos of test results may be labeled in advance, bounding boxes defined for the module to detect and crop up the result area throughout all the photos using a technique called Object Detection in the training phase. Further analysis is made by using computer vision technology to evaluate the result, whether a negative, positive or invalid result has been obtained.

The model called Yolo (You Only Look Once) may be used instead of CNN-based Object Detectors, because Yolo requires few computational resources whereas training a CNN requires more. The network architecture of Yolo consists of three parts: (1) Backbone: CSPDarknet, (2) Neck: PANet, and (3) Head: Yolo Layer. The data is first imputed to CSPDarknet for feature extraction, and then fed to PANet for feature fusion. Finally, a Yolo Layer outputs detection results (class, score, location, size).

A Behavioral Conformity Module (BCM) may be used to identify misuse of the test kit. For example, BCM may provide an algorithm based on deep learning for establishing a Behavioral Conformity Score according to actions made by the user while using a digital service. For example, the number of completed tasks, amount of process failures, dropping out of an active task etc. The algorithm provides an authenticity threshold value for accumulated user actions and alerts moderators of abnormal user behavior so that a submitted request can be flagged for an operator review and validated to make sure services are used properly and no foul play suspected.

A camera associated with the computing device, may obtain an image of an optical label present on one or more components of the test kit, the optical label containing identification information of the test kit. The computing device may identify the test kid based on the obtained image and a reference dataset which dataset may be remote.

As shown in FIG. 5F, the test kit may be adapted to produce a visual indication of a result that can be read by the device. The device camera may acquire an image comprising a visual representation of a test result and of the optical label present on a component of the test kit. The device processor, having confirmed the identity of the subject, and having determined that the test kit is the identified test kit based on the obtained image and the reference dataset, may then provide verification of the successfully completed self-test by sending, by the computing device, to an authorized party, the obtained image comprising the visual representation of the test result and of the optical label present on the component of the test kit. The information may be tabulated on a standardized form for sharing across a population of users of the device.

The system may utilize an antigen test kit, a serological test kit, a polymerase chain reaction (PCR) test kit, or other kit known in the art. In embodiments, the test kit comprises a lateral flow test strip forming a visible band on the strip when analyte is detected. A processor may be configured to evaluate an image of the trip to determine a test result. In one embodiment, the system and method according to the invention are implemented in a kiosk environment. The kiosk may provide a scanner adapted to scan documentation presented by a subject, such as government-issued identification (driver's license, passport or the like). The processor may transmit data to and receive data from a remote database to compare data obtained from the scan with a pre-stored data set of demographic information. The kiosk may provide a camera for obtaining a facial image of the subject. This information, together with the demographic information input by a subject into the computing device, or obtained from a scan of the scanned identification, may be used to verity the subject's identity. The test kit may be dispensed at the kiosk and the proper completion of the test may be monitored by camera. For example, and not by way of limitation, a test kit may require a nasal swab to be inserted to a nasal passage. The processor may be configured to identify indicia on the swab and determine position of the indicia relative to facial landmarks identified on the subject.

In another embodiment, the system and method according to the invention are implemented by an “app” or program stored in a non-transitory storage medium on a computing device such as a smartphone or laptop, containing instructions for a computing device to: receive input from a user, such as demographic information input by the user, data extracted from a scanned government-issued documentation of the user, and/or obtaining a facial image of the user; verify a test kit and the subject using the test kit; verify that the user is correctly using the test kit; and obtaining an image of the test kit after the self-test has been performed and determining a test result based on the image.

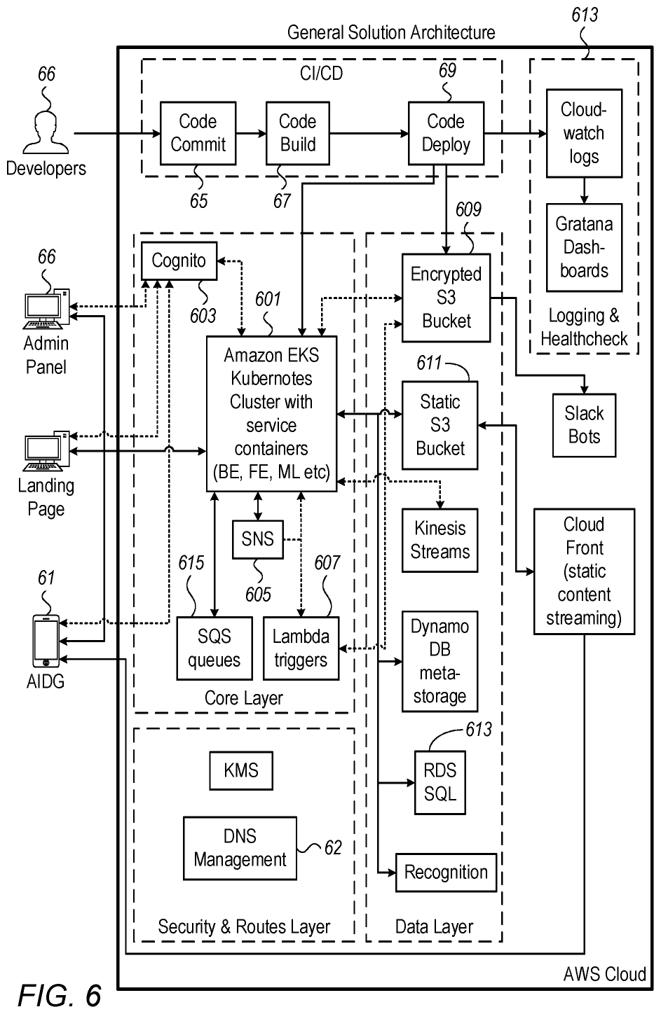

FIG. 6 is a schematic diagram of general system architecture according to an embodiment of the invention. The architecture may comprise two entry points for users: a mobile app 61 for patients and a website for Admin Panel operators 63. Entry point domains are managed with Route 53 DNS management 62.

The mobile application is made in React Native, the code is stored in a code commit 65. Assembly 67 and deployment 69 are carried out manually by the application developers 66. The back-end is written in Node JS, and the code is similarly stored in a code commit, and the assembly and deployment of the solution are carried out by an automatic pipeline using code build and code deploy.

The Admin Panel 63 is a website that may be divided into front-end and back-end containers. The front-end of the Admin Panel 63 may run on React JS, on a load-balanced Kubernetes cluster 601. The back-end may be written in Java and may run on a load-balanced Kubernetes cluster 601. The Admin Panel code is stored in a code commit, the assembly and deployment of the solution is carried out by an automatic pipeline using code build and code deploy.

The developers interact only with the GIT repository and can only make changes to the develop branch and merge to the staging branch.

Authorization at entry points is carried out using Cognito 603. The personal data of users and operators of the Admin Panel 63 is also stored there. Cognito 603 is used to create new accounts and monitor security events by security experts. For authorization, a two-factor authentication system is used, messages are sent using SNS 605 and Lambda services 607.

The Machine Learning container is written in Python, uses OpenCV for image pre-processing, Rekognition for document and face recognition, custom Yolo v5 models for automatic recognition of the correctness of the antigen test and the results. Models are stored in S3. The machine learning container code is stored in a code commit, the assembly and deployment of the solution is carried out by an automatic pipeline using code build and code deploy.

The solution uses double-encrypted S3 buckets 609, 611 to store images, large files, and machine learning models. Encryption is carried out with a KMS key at the bucket level, as well as customer SSE keys at the back-end level. The front-end does not have direct access to the buckets, all interactions with data are carried out at the level of the back-end business logic and the assembly and deployment pipelines of the solution.

The back-end components of the mobile application and the administrative panel use the DynamoDB document base with built-in encryption (separate for each back-end), it stores the metadata of the paths of the bucket files, as well as logical relationships and relationships between entities, identifiers. Daily backups are set.

The back-end of the mobile application uses RDS PostgreSQL 613 to store some auxiliary entities for the geolocation (names of countries, cities, languages, etc.), business logic data and personal data are not stored there.

All back-end containers receive messages generally in two ways—interaction at the API level and asynchronously using encrypted queues through the SQS message broker 615. The queue message contains database identifiers, S3 bucket file metadata, and technical instructions for business logic. Messages do not contain personal data and are stored in queues in encrypted form. Also, in addition to the main queues, deadletter queues are used, where back-end containers send messages with errors in the structure or headers for debugging.

The back-end and machine learning containers periodically check their SQS queues for new messages and process them as new messages appear.

There are generally at least two ways to exchange SQS messages:

Mobile application back-end→SQS MLE→MLE→SQS back-end admin panel→Administration panel back-end→SQS mobile application back-end

Mobile application back-end→SQS back-end admin panel→Administration panel back-end→SQS mobile application back-end

APIs may be used on back-end containers to interact primarily with front-end containers and the mobile app.

Logging and monitoring may be carried out using Cognito—authorization events; Cloud Watch—event logging in front-end, back-end containers, machine learning, logging and monitoring of developer actions in the AWS Console. Log data from back-end, front-end and ML is available for debug and development purposes on a separate instance with Grafana installed on it. Data about the start and end of the build and deployment pipelines is displayed in Slack using a bot.

Different embodiments are disclosed herein. Features of certain embodiments may be combined with features of other embodiments; thus, certain embodiments may be combinations of features of multiple embodiments. The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of verifying a self-test by a subject using a test kit, the method comprising:
obtaining, by a computing device, data identifying of the subject;
obtaining, by the computing device, data identifying the test kit;
verifying, by the computing device, that the identified subject is the subject utilizing the test kit;
obtaining, by the computing device, an in-process image of the self-test being performed and determining from the in-process image that the self-test has been properly performed;
obtaining, by the computing device, a results image of the self-test after the self-test has been performed and from said results image obtain a test result; and
registering in memory, by the computing device, the obtained test result.

2. The method of claim 1, wherein identifying the subject comprises:
obtaining, by a camera associated with the computing device, a first image of an identification document comprising a visual representation of a face of the subject,
obtaining, by the camera, a second image of the subject; and
identifying, by the computing device, the subject based on a comparison of the first image and the second image.

3. The method of claim 2, further comprising verifying, by the computing device, the identification document based on a comparison of data extracted from the first image of the identification document and a reference dataset.

4. The method of claim 3, wherein verifying that the identified subject is the subject utilizing the test kit comprises:
obtaining, by the camera, a third image comprising a visual representation of the face of the subject and of a component of the test kit, and
determining, by the computing device, that the identified subject is the subject utilizing the test kit based on the third image and at least one of the first image, the second image, the reference dataset, or any combination thereof.

5. The method of claim 4, further comprising verifying, by the computing device, that the subject is properly utilizing the test kit.

6. The method of claim 5, wherein verifying that the subject is properly utilizing the test kit comprises:
detecting, by the computing device, a specified location on the face of the subject in the third image,
detecting, by the computing device, a position of the component of the test kit with respect to the specified location on the face of the subject in the third image, and
determining, by the computing device, that the subject is properly utilizing the test kit based on the position of the component of the test kit with respect to the specified location on the face of the subject in the third image.

7. The method of claim 1, wherein obtaining data identifying the test kit comprises:
obtaining, by a camera associated with the computing device, a third image of an optical label present on one or more components of the test kit, the optical label containing identification information of the test kit, and
identifying, by the computing device, the test kit based on the obtained image and a reference dataset.

8. The method of claim 7, wherein obtaining the test result from the test kit and verifying that the test kit is the identified test kit comprising:
obtaining, by the camera, a fifth image comprising a visual representation of a test result and of the optical label being presented on a component of the test kit, and
determining, by the computing device, that the test kit is the identified test kit based on the obtained image and the reference dataset.

9. The method of claim 8, wherein registering the obtained test result with the authorized third-party comprising sending, by the computing device, to the authorized party the obtained image comprising the visual representation of the test result and of the optical label being presented on the component of the test kit.

10. A method of self-diagnosing of a subject using a test kit comprising a sample collecting tool and a testing tool, the method comprising:
obtaining, by a camera associated with a computing device, a first image of an identification document comprising a visual representation of a face of the subject,
obtaining, by the camera, a second image comprising a visual representation of the face of the subject,
identifying, by the computing device, the subject based on the first image and the second image,
obtaining, by the camera, a third image of an optical label being presented on one or more components of the test kit, the optical label containing identification information of the test kit,
identifying, by the computing device, the test kit based on the third image and a reference dataset,
obtaining, by the camera, a fourth image comprising a visual representation of the face of the subject and of the sample collecting tool of the test kit,
determining, by the computing device, that the identified subject is the subject utilizing the sample collecting tool of the test kit based on the fourth image and at least one of the first image or the second image,
detecting, by the computing device, a specified location on the face of the subject in the fourth image,
detecting, by the computing device, a position of the sample collecting tool of the test kit with respect to the specified location on the face of the subject in the fourth image,
determining, by the computing device, that the identified subject is properly utilizing the sample collecting tool of the test kit based on the position of the sample collecting tool with respect to the specified location on the face of the subject in the fourth image,
obtaining, by the camera, a fifth image comprising a visual representation of a test result and of the optical label being presented on the testing tool of the test kit,
determining, by the computing device, that the testing tool belongs to the identified test kit based on the fifth image and the reference dataset, and
registering the test result with an authorized third party by the computing device.

11. The method of claim 1, wherein the test kit is an antigen test kit.

12. The method of claim 1, wherein the test kit is a serological test kit.

13. The method of claim 1, wherein the test kit is a polymerase chain reaction (PCR) test kit.

14. A system for verifying a self-test by a subject, comprising:
a test kit adapted to provide a readable display of test results; and
a computing device having a processor configured to
obtain identifying information from a subject to verify the subject's identity;
obtain identifying information from the test kit to verify the test kit and associate the test kit with the subject;
obtain information about performance of the test kit; and
read the readable display of test results of the test kit.

15. The system according to claim 14, wherein
the test kit includes a bar code readable by the computing device to verify the test kit.

16. The system according to claim 14, wherein the computing device comprises a scanner adapted to scan government issued documentation of the subject to extract identifying information of the subject, and/or an image of the subject.

17. The system according to claim 14, wherein the processor is configured to compare the identifying information obtained from the subject with a remote dataset.

18. The system according to claim 14, wherein the computing device comprises a camera adapted to obtain an image of the subject's face, and wherein identifying information from a subject comprises facial features of the subject identified in the image of the subject's face.

19. The system according to claim 14, wherein the computing device comprises a camera adapted to obtain an image of the subject's face during performance of the test and verify correct performance of the test from an image of the subject's face during performance of the test.

20. The system according to claim 14, wherein the test kit comprises a lateral flow test strip forming a visible band on the strip when analyte is detected and the processor is configured to determine a positive or negative a test result from an image of the test strip.

21. The system according to claim 14, wherein the teste kit comprises a nasal swab and the processor is configured to identify indicia on the nasal swab in relation to facial features of the subject when the self-test is being performed.

22. The system according to claim 14, wherein the computing device is smart phone.

23. The system according to claim 14, wherein the computing device and test kit are associated in a kiosk location.
